(19) United States
(12) Patent Application Publication
(10) Pub. No.: US 2010/0130734 A1
(43) Pub. Date: May 27, 2010

(54) PROCESS FOR PREPARING CAPECITABINE

(75) Inventors: Raghavendracharyulu Venkata Palle, Hyderabad (IN); Anant Madhavrao Marathe, Hyderabad (IN); Srinivas Aluru, Hyderabad (IN); Ramesh Bochha, Hyderabad (IN); Rajasekhar Kadaboina, Secunderabad (IN); Sekhar Munaswamy Nariyam, Hyderabad (IN); Anil Patri, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad, Andhra Pradesh (IN); DR. REDDY'S LABORATORIES, INC., Bridgewater, NJ (US)

(21) Appl. No.: 12/596,544

(22) PCT Filed: Apr. 17, 2008
(86) PCT No.: PCT/US08/60573

§ 371 Date: Oct. 19, 2009

Related U.S. Application Data
(60) Provisional application No. 61/030,827, filed on Feb. 22, 2008.

(30) Foreign Application Priority Data
Apr. 20, 2007 (IN) 850/CHE/2007

(51) Int. Cl. C07H 19/06

(52) U.S. Cl. 536/28.51

(57) ABSTRACT

There is provided processes for the preparation of capecitabine and intermediates thereof.

### TECHNICAL FIELD

The present patent application relates to processes for the preparation of Capecitabine. Further, this application also relates to process for the preparation of intermediates of capecitabine.

### BACKGROUND

Capecitabine is chemically described as 5′-deoxy-5-fluoro-N-[(pentyloxy) carbonyl]-cytidine, represented by the chemical structure of Formula I.

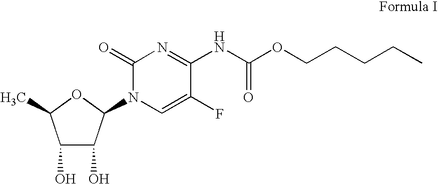

Capecitabine is a fluoropyrimidine carbamate with antineoplastic activity and is commercially available in the market under the brand name XELODA®.

Fuziu et al., in U.S. Pat. No. 4,966,891 disclose Capecitabine generically and a process for the preparation thereof. It also disclose the pharmaceutical composition, and method of treating of sarcoma and fibrosarcoma.

Kamiya et al., in U.S. Pat. No. 5,453,497 describes a process for the preparation of capecitabine, the process comprising the reaction of 5-deoxy-1,2,3-tri-O-acetyl-β-D-ribofuranose with a silylated 5-fluorocytosine using anhydrous stannic chloride, in methylene chloride.

Arasaki et al., in U.S. Pat. No. 5,472,949 discloses capecitabine specifically and a process for the preparation of 2′,3′-O-acetyl-5′-deoxy-N-[(pentyloxy)carbonyl-5-fluorocytidine, which is useful in the preparation of capecitabine, comprising the reaction of 2′,3′-O-acetyl-5′-deoxy-5-fluorocytidine with n-pentyl chloroformate.

Erion et al., in WO 94/18215 discloses protection of 2,3 hydroxyl groups of pentose sugars like L-lyxose into corresponding 2,3 isopropylidene derivative using methods known for 1,2 diol protection in the prior arts.

Jinliang et al, in WO 2005/0080351 discloses a process for the preparation of Capecitabine. This application also provides a process for acylation at the nitrogen atom in 5-fluoro cytosine with n-pentyl chloroformate to form N-[(pentyloxy)carbonyl]-5-fluorocytosine.

Carbohydrate Research 338 (2003) pages 303-306 discloses synthesis of 1,2,3-tri-O-acetyl-5-deoxy-D-ribofurnaose from D-ribose. This journal also discloses a process for the preparation of 2,3-isopropylidene-D-ribose from ribose using a catalytic amount of concentrated sulfuric acid.

Journal of American Chemical Society, 1955, volume 77, pages 2210-2212 discloses a process for the preparation of the intermediate compound 5-deoxy-D-ribose, which is useful in the preparation of Capecitabine.

Journal of Medicinal Chemistry, 1977, volume 20, pages 344-348 discloses a process for the preparation of 2′,3′-O-isopropylidene-5-fluorocytidine, which is useful in the preparation of Capecitabine, comprising a reaction of 5-fluorocytosine with 2,2-dimethoxypropane.

In view of plethora of disclosures for processes of intermediates and Capecitabine, it is apparent that, there is still a need for convenient processes for the preparation of Capecitabine as well as its intermediates with desired purity and yield using improved preparation techniques, which may be used for the commercial manufacturing.

### SUMMARY

The present invention provides processes for the preparation of Capecitabine and intermediates thereof.

In one aspect, the present invention provides processes for the preparation of 5′-deoxy-2′,3′-O-isopropylidene-N-[(pentyloxy)carbonyl]-5-fluorocytidine of Formula II

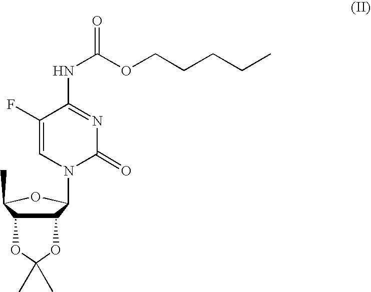

which process comprises:
a) reacting 5-deoxy-D-ribose of Formula V:

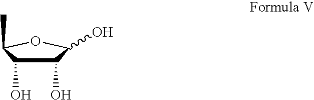

with 2,2-dimethoxypropane in the presence of a suitable organic solvent to afford the compound 2,3-O-isopropylidene-5-deoxy-D-ribose of Formula IV;

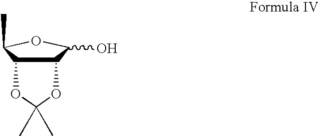

b) reacting the compound 2,3-O-isopropylidene-5-deoxy-D-ribose of Formula IV
with acetic anhydride in the presence of a suitable organic solvent to afford the compound 2,3-O-isopropylidene-3-O-acetyl-5-deoxy-D-ribose of Formula III

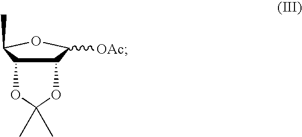

and
c) reacting the compound 2,3-O-isopropylidene-3-O-acetyl-5-deoxy-D-ribose of Formula III with the 2-O-trimethyl silyl N-[(pentyloxy)carbonyl]-5-fluorocytosine of Formula IIIB:

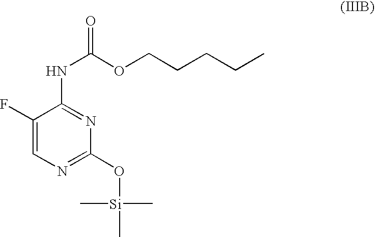

in the presence of a suitable organic solvent to afford the compound 5′-deoxy-2′,3′-O-isopropylidene-N-[(pentyloxy)carbonyl]-5-fluorocytidine of Formula II.

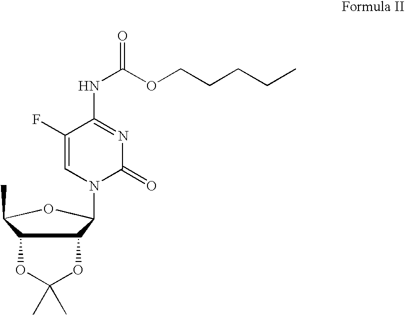

In another embodiment, there is provided another process for the preparation of 5′-deoxy-2′,3′-O-isopropylidene-N-[(pentyloxy)carbonyl]-5-fluorocytidine of Formula II, which process comprises:
i) reacting the compound 2,3-O-isopropylidene-1-O-acetyl-5-deoxy-D-ribose of Formula III:

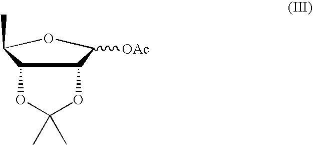

with the compound of Formula IIIC:

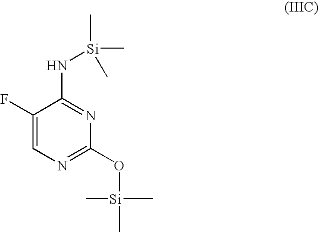

in the presence of stannic chloride and a suitable organic solvent to afford the compound 5′-deoxy-2′,3′-O-isopropylidene-5-fluorocytidine of Formula IV

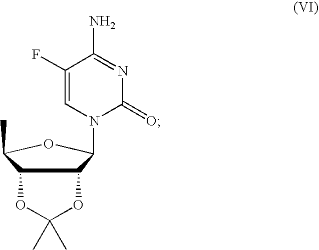

and
ii) reacting the compound 5′-deoxy-2′,3′-O-isopropylidene-5-fluorocytidine of Formula VI with n-pentyl chloroformate in the presence of a suitable organic solvent to afford 5′-deoxy-2′,3′-O-isopropylidene-N-[(pentyloxy)carbonyl]-5-fluorocytidine of Formula II.

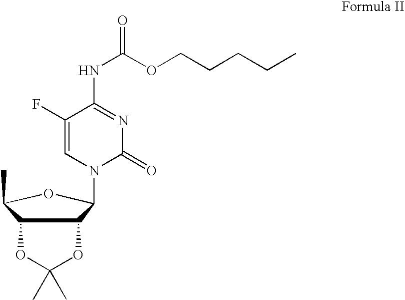

In another aspect, there is provided processes for the preparation of capecitabine of Formula I.

In one embodiment of the present invention provides a process for preparing Capecitabine comprises by converting Formula II or Formula C to capecitabine, wherein the conversion is preceded by deprotection, also referred to herein as selective deprotection. When the conversion is from a protected molecule wherein both the —OH groups in the 2′ and 3′ positions of the ribose ring are protected in the manner illustrated by Formula II, said selective deprotection is carried out with Amberlyst™ 15 catalyst. The selective deprotection is selective for deprotection at position 2′ and 3′ of the compound of formula-II.

In another embodiment of the present invention provides improved process for preparing Capecitabine, which process comprises:
a) reacting 5′-deoxy-2′,3′-O-acetyl-5-fluorocytidine of Formula A

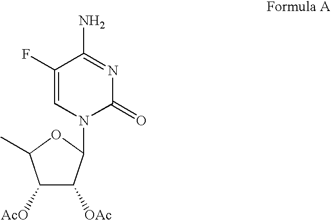

with n-pentyl chloroformate of Formula B

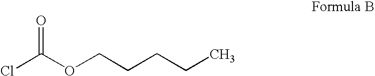

in the presence of pyridine and organic solvent to form 5′-deoxy-2′,3′-β-acetyl-N-[(pentyloxy)carbonyl]-5-fluorocytidine of Formula C

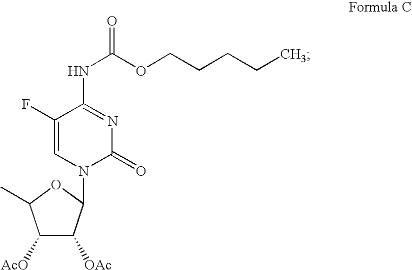

and
b) deprotection of hydroxyl protecting groups of Formula C using base such as sodium hydroxide in the presence of methanol to form Capecitabine of Formula I.

In yet another aspect the present invention provides a process for the preparation of the intermediate -2-O-trimethyl silyl N-[(pentyloxy)carbonyl]-5-fluorocytosine of Formula IIIB

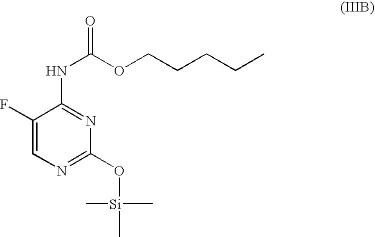

which process comprising reacting the compound N-[(pentyloxy)carbonyl]-5-fluorocytosine of Formula IIIA

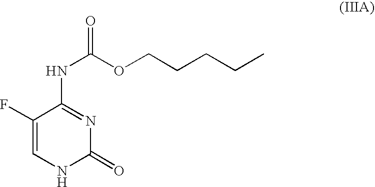

with a suitable silylated reagent.

The other embodiments of the invention there are provided methods of making crystalline forms of capecitabine, crystalline capecitabine, processes of milling crystalline capecitabine, processes of making capecitabine in different crystalline forms, as well as capecitabines of differing particle size distributions. The invention also includes a capecitabine having a PXRD as shown substantially in FIG. 1, as well as a capecitabine having a PXRD as shown substantially in FIG. 5. These aspects include a process for the preparation of compound of Formula I

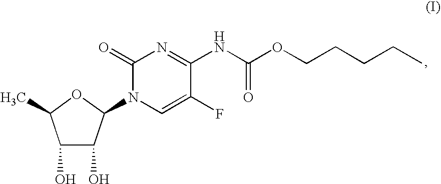

comprising deprotecting a compound of Formula I wherein the OH groups in positions 2′ and 3′ are protected.
In a particularly preferred aspect of this process, the compound of Formula I, wherein the OH groups in positions 2′ and 3′ are protected, has the structure of the compound of Formula II

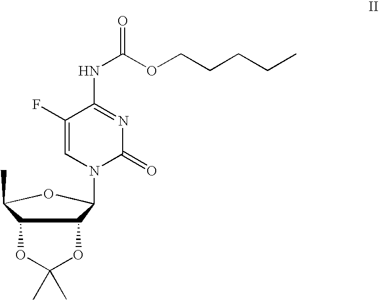

or the structure of the compound of Formula C

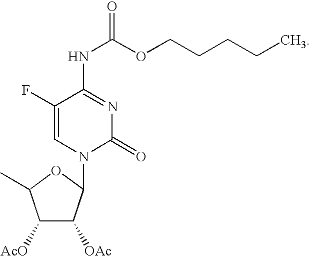

The compound of Formula I, produced from deprotecting the compound of Formula II, can be isolated by crystallization comprising dissolving the reaction mixture in a solvent followed by cooling of the solution.
The compound Formula I may be characterized by X-ray powder diffraction pattern (XRPD) substantially in accordance with FIG. 1.
Another aspect of the invention is a process for preparing micronized Capecitabine, which comprises the milling of crystalline material of Capecitabine in micronizer at set feeding pressure of about 2 Kgs/cm² to about 5 Kgs/cm². The resulting micronized Capecitabine is characterized by X-ray powder diffraction pattern (XRPD) substantially in accordance with FIG. 5.
In other aspects, the invention recites a process for preparing the compound of Formula II,

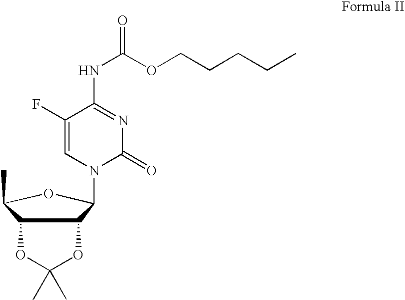

comprising:
a) reacting compound 5-deoxy-D-ribose of Formula V:

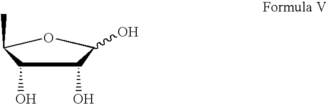

with 2,2-dimethoxypropane in an organic solvent to afford 2,3-O-isopropylidene-5-deoxy-D-ribose of Formula IV;

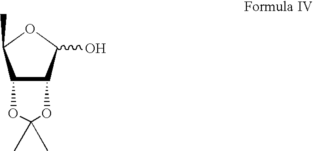

b) reacting the compound 2,3-O-isopropylidene-5-deoxy-D-ribose of Formula IV with acetic anhydride in the presence of an organic solvent to afford 2,3-O-isopropylidene-1-O-acetyl-5-deoxy-D-ribose of Formula III

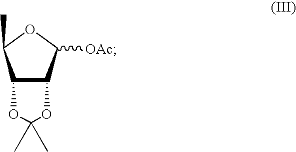

and
c) reacting the compound 2,3-O-isopropylidene-1-O-acetyl-5-deoxy-D-ribose of Formula III with 2-O-trimethyl silyl, N-[(pentyloxy)carbonyl]-5-fluorocytosine of Formula IIIB:

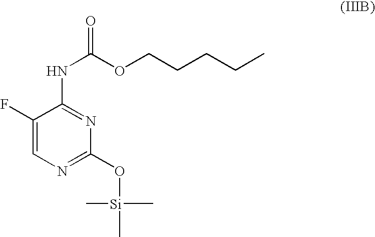

in the presence of an organic solvent to afford 5′-deoxy-2′,3′-O-isopropylidene-N-[(pentyloxy)carbonyl]-5-fluorocytidine of Formula II.
In still another aspect, the invention provides a process for preparing intermediate-5′-deoxy-2′,3′-O-isopropylidene-N-[(pentyloxy)carbonyl]-5-fluorocytidine of Formula II, comprising:
i) reacting the compound 2,3-O-isopropylidene-3-O-acetyl-5-deoxy-D-ribose of Formula III:

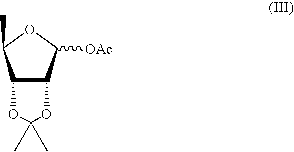

with 2-O-trimethyl silyl, N-(trimethyl silyl)-5-fluorocytosine of Formula IIIC:

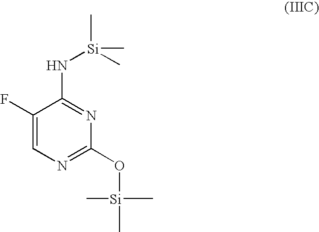

in the presence of stannic chloride and an organic solvent to afford the compound 5′-deoxy-2′,3′-O-isopropylidene-5-fluorocytidine of Formula VI

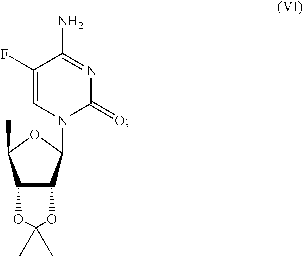

and
ii) reacting the compound 5′-deoxy-2′,3′-O-isopropylidene-5-fluorocytidine of Formula VI with n-pentyl chloroformate in the presence of an organic solvent to afford the compound of Formula II.
Another embodiment is micronized Capecitabine obtained having particle size distribution of D₉₀ less than about 25 microns and D₅₀ less than about 15 microns.
This micronized Capecitabine may have an X-ray powder diffraction pattern (XRPD) substantially in accordance with FIG. 5.

### BRIEF DESCRIPTION OF THE DRAWINGS

FIG. 1 shows an illustrative example of X-ray powder diffraction pattern of Capecitabine (before Micronization) prepared according to Example 9.

FIG. 2 shows an illustrative example of differential scanning calorimetry curve of Capecitabine (before Micronization) prepared according to Example 9.

FIG. 3 shows an illustrative example of thermogravimetric analysis curve of Capecitabine (before Micronization) prepared according to Example 9.

FIG. 4 shows an illustrative example of Polarising light microscopy image of Capecitabine (before Micronization) prepared according to Example 9.

FIG. 5 shows an illustrative example of X-ray powder diffraction pattern of Capecitabine (after Micronization) prepared according to Example 9.

FIG. 6 shows an illustrative example of Polarising light microscopy image of Capecitabine (after Micronization) prepared according to Example 9.

### DETAILED DESCRIPTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims.

The term “compound” is used to refer to a molecular entity of defined chemical structure.

The term “solvent” defines any liquid medium in which component(s) is/are dissolved, including an individual solvent or a mixture of solvents.

Amongst other things, with reference to the powder X-ray diffraction patterns (PXRD) provided, this document may state that a material has the PXRD pattern “substantially” as shown, “substantially” in accordance, or other words to that effect. In those instances, it will be appreciated that PXRD patterns may be shifted, in whole or in part, due to a number of factors understood by those in the field. These factors include, without limitation, differences in equipment, differences in technique, differences in calibration, differences in sample preparation, run times and error. These factors may also affect the relative peak intensities. The term “substantially” was used to allow for such variations. If a person of ordinary skill in the field of PXRD can look at the figures and a pattern of an unknown form of capecitabine and evaluate whether or not they are in fact the same form, that is sufficient to fall within the description and claims. The present invention provides a process for the preparation of capecitabine and intermediates thereof.

In one aspect, there is provided processes for preparing the compound 5′-deoxy-2′,3′-O-isopropylidene-N-[(pentyloxy)carbonyl]-5-fluorocytidine of Formula II.

In one embodiment, there is provided a process for the preparation of the compound of Formula II, which process comprises:
a) reacting 5-deoxy-D-ribose of Formula V:

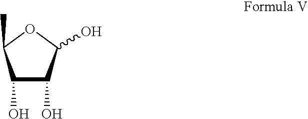

with 2,2-dimethoxypropane in the presence of a suitable organic solvent to afford the 2,3-O-isopropylidene-5-deoxy-D-ribose of Formula IV;

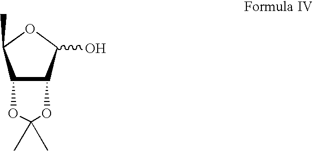

b) reacting the compound 2,3-O-isopropylidene-5-deoxy-D-ribose of Formula IV with acetic anhydride in the presence of a suitable organic solvent to afford the compound 2,3-O-isopropylidene-3-O-acetyl-5-deoxy-D-ribose of Formula III

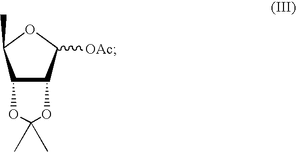

and
c) reacting the compound 2,3-O-isopropylidene-3-O-acetyl-5-deoxy-D-ribose of Formula III with 2-O-trimethyl silyl, N-[(pentyloxy)carbonyl]-5-fluorocytosine of Formula IIIB:

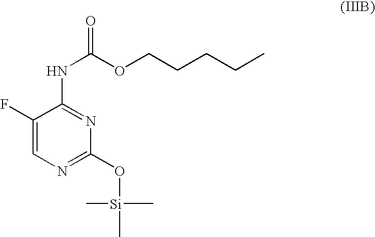

in the presence of a suitable organic solvent to afford the Intermediate compound -5′-deoxy-2′,3′-O-isopropylidene-N-[(pentyloxy)carbonyl]-5-fluorocytidine of Formula II.

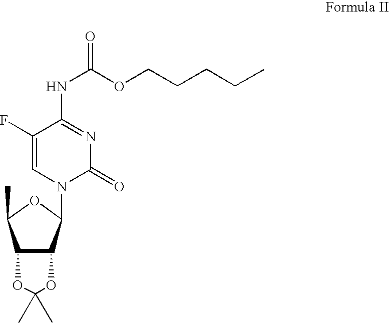

Overall process can be summarized in the scheme mentioned herein below:

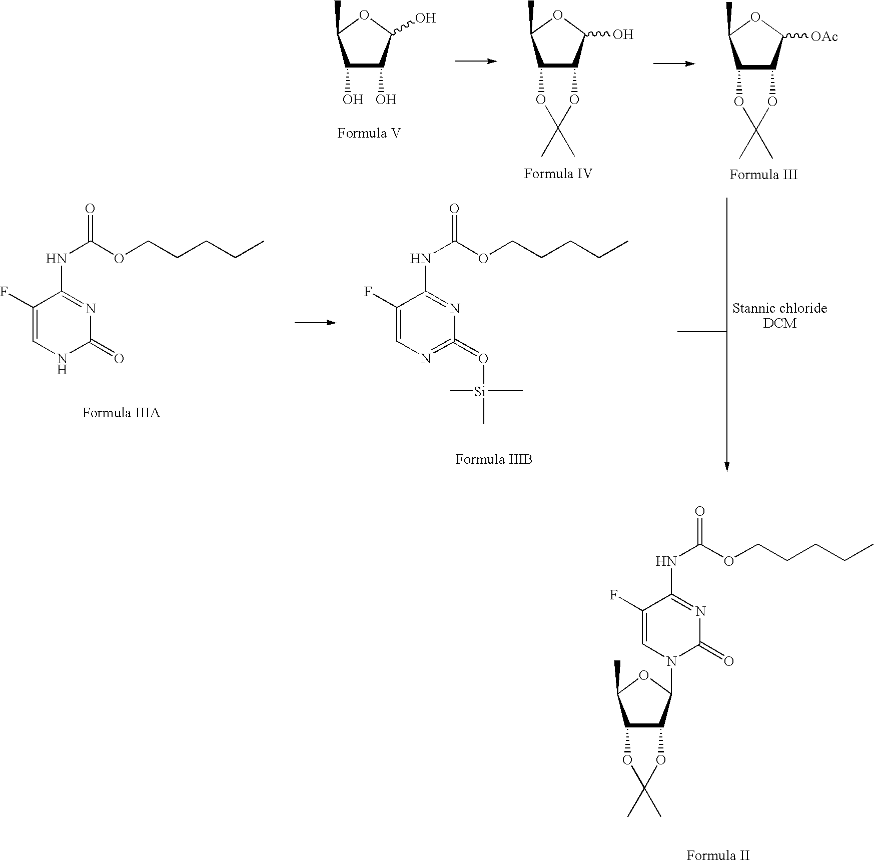

Step a) involves reacting —OH groups at the 2 and 3 positions in the compound 5-deoxy-
D-ribose of Formula V with 2,2-dimethoxypropane in the presence of a suitable organic solvent to afford the compound 2,3-O-isopropylidene-5-deoxy-D-ribose of Formula IV. The protection of hydroxyl groups are conducted selectively.
This protection step may be carried out in the presence of an acid. Acids which can be used for the selective protection of —OH groups include but are not limited to inorganic acids such as hydrochloric acid, sulphuric acid, and the like; and organic acids such as oxalic acid, tartaric acid, formic acid, acetic acid, and para-toluene sulfonic acid.
The temperature and time may be dependent on many factors such as the choice of acid used, and the amount of starting material. The temperature may be range from about 0 to about 50° C., or higher. The time period to achieve the desired product yield and purity, times from about 1 to 20 hours, or longer, frequently being adequate.
Suitable organic solvents which can be used to carry out the protection include, but are not limited to: halogenated solvents such as dichloromethane, 1,2-dichloroethane, chloroform, carbon tetrachloride and the like; esters such as ethyl acetate, n-propyl acetate, n-butyl acetate, t-butyl acetate and the like; ether solvents such as diethyl ether, dimethyl ether, di-isopropyl ether, methyl tertiary-butyl ether, tetrahydrofuran, 1,4-dioxane and the like; hydrocarbon solvents such as toluene, xylene, heptane, hexane and the like; N,N-dimethylformamide (DMF), dimethyl sulfoxide (DMSO), and the like; and mixtures thereof.
After completion of the reaction, the product may be recovered from the reaction mixture by any means known in the art.
   Step b) involves reacting the compound 2,3-O-isopropylidene-5-deoxy-D-ribose of Formula IV with acetic anhydride in the presence of a suitable organic solvent to afford the compound 2,3-O-isopropylidene-3-O-acetyl-5-deoxy-D-ribose of Formula III.
The reaction can be carried out under basic conditions using a suitable base. Bases that can be used include but are not limited to: organic bases such as pyridine, triethylamine, and methylamine; and inorganic bases such as sodium hydroxide, potassium hydroxide, and lithium hydroxide.

The reaction temperature can range from about −25 to about 60° C., or higher.

Step b) can be carried out in the presence or absence of a solvent.
Suitable organic solvents that can be used include but are not limited to: pyridine, triethylamine, methylamine, dichloromethane, chloroform, and carbon tetrachloride; hydrocarbon solvents such as toluene, xylene, heptane, and hexane; and esters such as ethyl acetate, n-propyl acetate, n-butyl acetate, and t-butyl acetate.
The reaction can be carried out for any desired time periods to achieve the desired product yield and purity, times from about 1 to 10 hours, or longer, frequently being adequate.
   Step c) involves reacting the compound 2,3-O-isopropylidene-3-O-acetyl-5-deoxy-D-
ribose of Formula III with the compound silylated N-[(pentyloxy)carbonyl]-5-fluorocytosine of Formula IIIB in the presence of a suitable organic solvent to afford the compound 5′-deoxy-2′,3′-O-isopropylidene-N-[(pentyloxy) carbonyl]-5-fluorocytidine of Formula II.
The reaction of step c) may be carried out using a catalytic amount of stannic chloride. Other catalysts that can be used include, but are not limited to, stannous chloride, trimethylsilyl trifluoromethanesulfonate, platinum, palladium or rhodium in concentrated sulfuric acid.
Suitable organic solvents which can be used include but are not limited to: chlorinated solvents such as dichloromethane, 1,2-dichloroethane, chloroform, carbon tetrachloride and the like; hydrocarbon solvents such as toluene, xylene, heptane, hexane and the like; and mixtures thereof. Suitable temperatures for conducting the reaction of step c) may range from about −20 to about 50° C., or higher.
After completion of the reaction, the reaction residue, which is obtained by the concentration of reaction mixture, comprising 5′-deoxy-2′,3′-O-isopropylidene-N-[(pentyloxy)carbonyl]-5-fluorocytidine of Formula II may be purified using a column chromatography technique or an anti solvent technique, or it can be purified using recrystallization in a suitable solvent.
Suitable solvents for the purification include but are not limited to: halogenated solvents such as dichloromethane, 1,2-dichloroethane, chloroform, carbon tetrachloride and the like; alcohols such as methanol, ethanol, isopropyl alcohol, and the like; esters such as ethyl acetate, n-propyl acetate, n-butyl acetate, t-butyl acetate and the like; hydrocarbon solvents such as toluene, xylene, heptane, hexane, petroleum ether, and the like; ether solvents such as diethyl ether, dimethyl ether, di-isopropyl ether, methyl tertiary-butyl ether, tetrahydrofuran, 1,4-dioxane and the like; ketone solvents such as acetone, methyl ethyl ketone and the like; N,N-dimethylformamide (DMF), dimethyl sulfoxide (DMSO), water and the like; and mixtures thereof. Indeed, these solvents can generally be used in any crystallization process described herein.
The reaction can be carried out for any desired time periods to achieve the desired product yield and purity, times from about 1 to 10 hours, or longer, frequently being adequate.

In another embodiment, there is provided another process for the preparation of the compound -5′-deoxy-2′,3′-O-isopropylidene-N-[(pentyloxy)carbonyl]-5-fluorocytidine of Formula II, which process comprises:
i) reacting the compound 2,3-O-isopropylidene-3-O-acetyl-5-deoxy-D-ribose of Formula III:

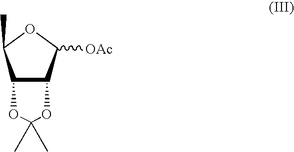

with the compound of Formula IIIC:

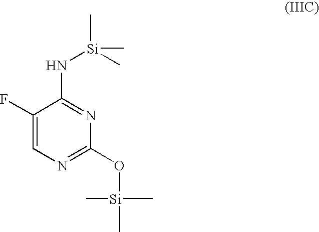

in the presence of stannic chloride and a suitable organic solvent to afford the compound 5′-deoxy-2′,3′-O-isopropylidene-5-fluorocytidine of Formula VI

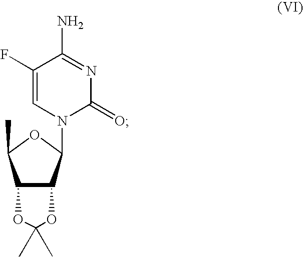

and
ii) reacting the compound 5′-deoxy-2′,3′-O-isopropylidene-5-fluorocytidine of Formula VI with n-pentyl chloroformate in the presence of a suitable organic solvent to afford the compound 5′-deoxy-2′,3′-O-isopropylidene-N-[(pentyloxy)carbonyl]-5-fluorocytidine of Formula II.

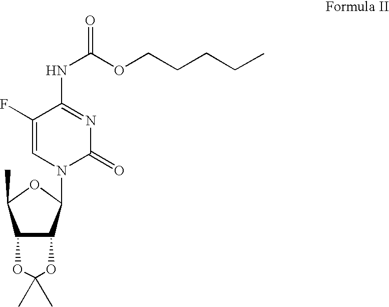

The overall process is summarized in the following scheme:

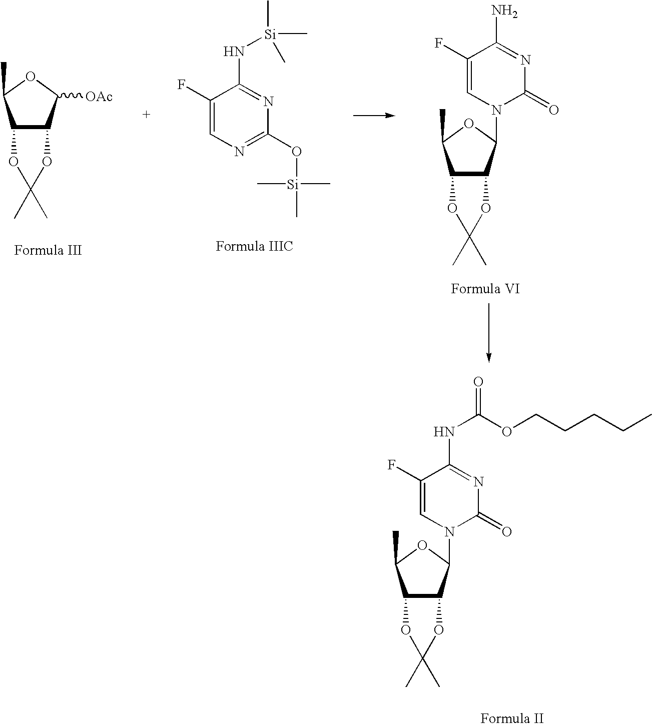

Step i) involves reacting the compound 2,3-O-isopropylidene-3-O-acetyl-5-deoxy-D-
ribose of Formula III with the compound silylated 5-fluorocytosine of Formula IIIC in the presence of stannic chloride and a suitable organic solvent under suitable conditions to afford the compound 5′-deoxy-2′,3′-O -isopropylidene-5-fluorocytidine of Formula VI.
The condensation reaction may be carried out with a catalytic amount of stannic chloride. Other catalysts such as stannous chloride, trimethylsilyl trifluoromethanesulfonate, platinum, palladium or rhodium in concentrated sulfuric acid, and the like are also useful for the condensation reaction.
The amount of stannic chloride is used in the reaction can be range from about 0.5 to about 2 molar equivalent per molar equivalent of the compound of Formula III.
Suitable organic solvents which can be used include but are not limited to: chlorinated solvents such as dichloromethane, 1,2-dichloroethane, chloroform, carbon tetrachloride and the like; hydrocarbon solvents such as toluene, xylene, n-heptane, hexane and the like; and mixtures thereof.
Suitable temperatures used in the condensation reaction are from about -20 to about 50° C., or higher.
The reaction can be carried out for any desired time periods to achieve the desired product yield and purity, times from about 1 to 10 hours, or longer, frequently being adequate.
After completion of the reaction, the reaction mixture comprising 5′-deoxy-2′,3′-O-isopropylidene-5-fluorocytidine of Formula VI may be purified using a column chromatography technique or an anti solvent technique, or it can be purified using recrystallization in a suitable solvent.
Suitable solvents for the purification include but are not limited to: halogenated solvents such as dichloromethane, 1,2-dichloroethane, chloroform, carbon tetrachloride and the like; alcohols such as methanol, ethanol, isopropyl alcohol, and the like; esters such as ethyl acetate, n-propyl acetate, n-butyl acetate, t-butyl acetate and the like; hydrocarbon solvents such as toluene, xylene, heptane, hexane, petroleum ether, and the like; ether solvents such as diethyl ether, dimethyl ether, di-isopropyl ether, methyl tertiary-butyl ether, tetrahydrofuran, 1,4-dioxane and the like; N,N-dimethylformamide (DMF), dimethyl sulfoxide (DMSO), water and the like; and mixtures thereof.
   Step ii) involves reacting the compound of 5′-deoxy-2′,3′-O-isopropylidene-5-fluorocytidine with n-pentyl chloroformate in the presence of a suitable organic solvent under suitable conditions to afford the compound 5′-deoxy-2′,3′-O-isopropylidene-N-[(pentyloxy)carbonyl]-5-fluorocytidine of Formula II.
The quantity of acylating agent such as n-pentyl chloroformate used for the formation of Formula II may range from about 1 to about 4 molar equivalents per molar equivalent of the compound of Formula VI.

The reaction of step ii) can be carried out in the presence or absence of solvent.
Suitable organic solvents that can used include but are not limited to: halogenated solvents such as dichloromethane, 1,2-dichloroethane, chloroform, carbon tetrachloride and the like; hydrocarbon solvents such as toluene, xylene, heptane, hexane, petroleum ether, and the like; ether solvents such as diethyl ether, dimethyl ether, di-isopropyl ether, methyl tertiary-butyl ether, tetrahydrofuran, 1,4-dioxane and the like; and mixtures thereof.
The reaction of step ii) can be carried out at temperatures ranging from about −30 to about 45° C., or from about −15 to about 0° C.
The reaction can be carried out for any desired time periods to achieve the desired product yield and purity, times from about 1 to 10 hours, or longer, frequently being adequate.
The reaction mixture comprising 5′-deoxy-2′,3′-O-isopropylidene-N-[(pentyloxy)carbonyl]-5-fluorocytidine of Formula II may be used directly in the next processing step or it can be concentrated to form a residue.

In yet another aspect, the present invention provides a process for the preparation of the compound of Formula IIIB, which is used as an intermediate in the preparation of Capecitabine. The process comprises reacting the compound N-[(pentyloxy)carbonyl]-5-fluorocytosine of Formula IIIA with suitable silylated reagent as per scheme mentioned below—

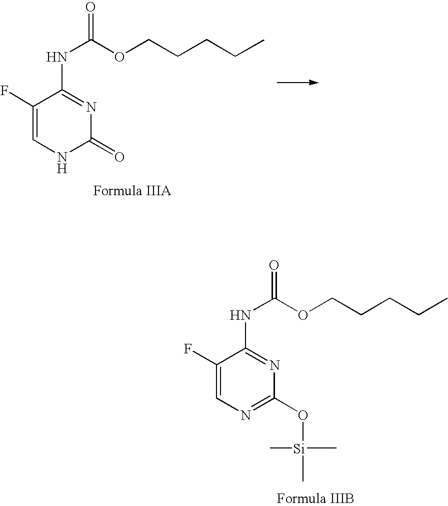

to afford the compound of Formula IIIB.

N-[(pentyloxy)carbonyl]-5-fluorocytosine of Formula IIIA of the present invention can be prepared through methods known in the art. For example, it can be prepared using the process disclosed in WO 2005/0080351 or it can be prepared by the reaction of 5-fluorocytosine with N-pentylchloroformate according to the process of the present invention.

Suitable silylating reagents that can be used include but are not limited to: hexamethyldisilazane (HMDS), hexamethyldisiloxane, methyltrichlorosilane, trimethylsilylchloride (TMS-CI), butyldimethylchlorosilane, tert-butyldimethylchlorosilane solution, dimethylchlorosilane, 1,1,3,3-tetramethyldisilazane and the like, and mixtures thereof.

The silylation reaction can be carried out in the presence or absence of a solvent.

Suitable solvents that can be used include but are not limited to: chlorinated solvents such as dichloromethane, 1,2-dichloroethane, chloroform, carbon tetrachloride and the like; esters such as ethyl acetate, n-propyl acetate, n-butyl acetate, t-butyl acetate and the like; hydrocarbon solvents such as toluene, xylene, heptane, hexane and the like; and mixtures thereof.

The reaction temperature for the silylation can range from about 20 to about 100° C., or higher.

The obtained reaction solution comprising silylated N-[(pentyloxy) carbonyl]-5-fluorocytosine may be directly used in the further processing step or it can be stripped using hydrocarbon solvents. Suitable hydrocarbon solvents that can be used include but are not limited to toluene, hexane, heptane, cyclohexane and the like.

The reaction can be carried out for any desired time period to achieve the desired product yield and purity, times from about 1 to 10 hours, or longer, frequently being adequate.

In another aspect, there are provided processes for the preparation of Capecitabine of Formula I suitable for large scale preparation.

In one embodiment of the present invention, which provides process for preparing capecitabine by converting Formula II, which is preferably prepared by any of the processes of the invention into Capecitabine, but which can be derived from any process, wherein the conversion is preceded by deprotection, also referred to as selective deprotection. The said selective deprotection is carried out with Amberlyst™ 15 catalyst.

The overall process is summarized in the following scheme:

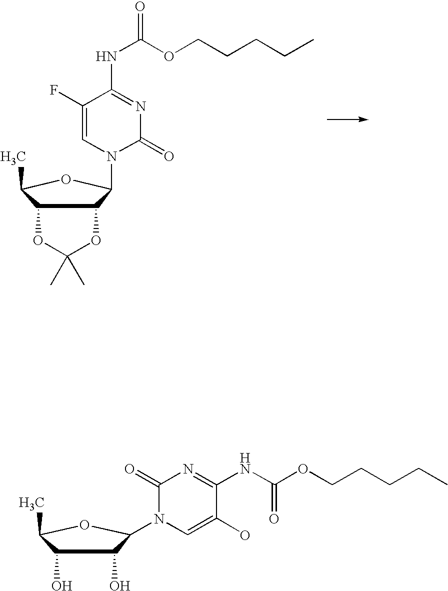

The inventors of the present invention have developed a new process for deprotection of protecting groups of protected Capecitabine selectively with readily available and cheaper reagent such as Amberlyst™ 15 catalyst, owing to recyclability.

Amberlyst 15 ion-exchange resin can be used in the form of dry or wet material for deprotection of protecting groups. The amount of catalyst may range from about 0.5 to about 2 times on the weight of the compound Formula II.

The deprotection reaction can be carried out in a solution, or in an aqueous suspension with or without the addition of an organic solvent. Suitable organic solvents that can be used are methanol, ethanol, isopropyl alcohol, n-butanol, and the like.

The reaction can be carried out at temperatures of about 20 to about 50° C., or from about 25 to about 35° C.

The reaction can be carried out for any desired time periods to achieve the desired product yield and purity, times from about 1 to 10 hours, or longer, frequently being adequate.

After completion of the reaction, the reaction mixture is filtered, and filtrate is concentrated completely under vacuum. The concentrated residue is dissolved in a suitable solvent selected from esters such as ethyl acetate, n-propyl acetate, n-butyl acetate, and t-butyl acetate; ether solvents such as diethyl ether, dimethyl ether, di-isopropyl ether, methyl tertiary-butyl ether, tetrahydrofuran, and 1,4-dioxane; hydrocarbon solvents such as toluene, xylene, heptane, and hexane; and mixtures thereof. Pure capecitabine is precipitated by cooling the solution to about −20 to about 0° C.

In another embodiment of the present invention provides a process for preparing capecitabine, which comprises:

a) reacting 5′-deoxy-2′,3′-O-acetyl-5-fluorocytidine of Formula A

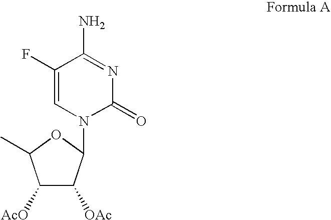

with n-pentyl chloroformate of Formula B

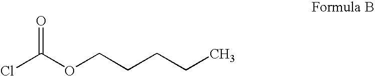

in the presence of pyridine and organic solvent to form 5′-deoxy-2′,3′-O-acetyl-N-[(pentyloxy)carbonyl]-5-fluorocytidine of Formula C

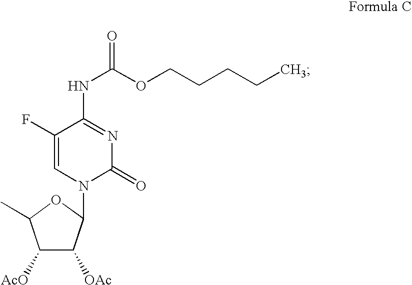

and

b) deprotection of hydroxyl protecting groups of Formula C using base such as sodium hydroxide in the presence of methanol to form Capecitabine of Formula I.

5′-deoxy-2′,3′-O-acetyl-5-fluorocytidine of Formula A is reacted with n-pentyl chloroformate of Formula B in the presence of base like pyridine and organic solvent. n-pentyl chloroformate is added slowly to the reaction mass at temperature less than 5° C. Suitably, the addition of n-pentyl chloroformate is carried out slowly range from about 30 minutes to 5 hours or more. The said reaction mass is formed by adding the compound of 5′-deoxy-2′,3′-O-acetyl-5-fluorocytidine of Formula A, pyridine and an organic solvent to a suitable reaction vessel.

The quantity of n-pentyl chloroformate is used for the formation of Formula C can be from about 1 to about 4 molar equivalents per molar equivalent of the compound of Formula A, preferably 2 to 3 molar equivalents.

The quantity of pyridine is used for the formation of Formula C may be from about 1 to about 4 molar equivalents per molar equivalent of the compound of Formula A, preferably 2 to 3 molar equivalents.

Organic solvent that is utilized in the reaction include but are not limited to: halogenated solvent such as dichloromethane, chloroform, dichloroethane, and chlorobenzene, preferably dichloromethane.

The temperature and time for conducting the reaction may be dependent on many factors such as the choice of base used, and the amount of starting material (Formula A). The temperature may be range from about −40 to about 40° C., or higher, preferably −15 to 5° C. The time period to achieve the desired product yield and purity, times from about 1 to 20 hours, frequently being adequate, preferably 1 to 2 hours.

After completion of the reaction, the reaction mixture is quenched with alcohol such as methanol, ethanol, isopropyl alcohol and n-propanol; and then the reaction mixture is diluted with the mixture of water and organic solvent. Further, the reaction mixture is extracted into an organic layer and then the organic layer is concentrated. Organic solvent is selected from dichloromethane, and chloroform.

Step b) deprotection of hydroxyl protecting groups of Formula C obtained from step a) using base such as sodium hydroxide in the presence methanol to form Capecitabine of Formula I. The reaction of step b) may be carried out at a temperature of about −30 to about 20° C. or more.

Amount of sodium hydroxide (1N NaOH) is about equimolar or more than equimolar to the Formula C, preferably 1 to 2 moles. Sodium hydroxide can be used as aqueous solution.

Preferably, the addition of the sodium hydroxide solution is carried out slowly to control the exothermicity of the reaction and to maintain the temperature of the reaction medium low, preferably, from less than about −20° C. to less than about 5° C. An increase in temperature may cause formation of side products and process-related impurities.

After completion of the reaction, the reaction mixture is extracted into organic solvent after adjusting the pH range from 3 to 6 with hydrochloric acid and then the obtained organic layer is concentrated completely to obtain crude. Organic solvent may be selected from dichloromethane and chloroform.

The solid may be isolated from the obtained crude containing Capecitabine of Formula I, by using solvent or mixture of solvents selected from ethyl acetate/n-hexane, ethyl acetate/n-heptane, acetone/n-heptane, dichloromethane/n-heptane, dichloromethane/toluene, ethyl acetate/toluene, acetone/demineralized water, acetone/methyl tertiary butyl ether, acetone/diisopropyl ether, acetone/toluene, dichloromethane/diisopropyl ether, and ethyl acetate.

The solid product may optionally be further dried. Drying can be suitably carried out in a tray dryer, vacuum oven, air oven, fluidized bed drier, spin flash dryer, flash dryer and the like. The drying can be carried out at temperatures of about 35° C. to about 90° C. with or without vacuum. The drying can be carried out for any desired time until the required product purity is achieved, time periods from about 1 to 20 hours, or longer, frequently being sufficient.

The capecitabine may be further purified using a column chromatography technique, a recrystallization technique, or a combination thereof.

Capecitabine prepared in accordance with the process of the present invention, in one embodiment, contains less than about 0.5%, or, in another embodiment, less than about 0.1%, by weight, of individual corresponding process or structural impurities as determined using high performance liquid chromatography (“HPLC”).

Capecitabine obtained by any process of the present application, unless stated otherwise, are characterized by their X-ray powder diffraction (“XRPD”) patterns, differential scanning calorimetry (“DSC”) curves, and thermogravimetric analysis (TGA) curves substantially as shown in the figures. “Substantially” has a meaning similar to that used in conjunction with PXRD patterns.

All XRPD data reported herein were obtained using a Bruker AXS D8 Advance Powder X-ray Diffractometer, equipped with Bragg-Berntano Φ:Φ goniometer. The pattern was recorded at a tube voltage of 40 kV and a tube current of 40 mA, with a step size of 0.013° and time per step of 0.1 sec over an angular range of 3-45° 2 theta. The sample was grounded gently and filled in a sample holder by top loading method and the sample was exposed to the Cu K-α radiations (wavelength 1.5406 Å). Since some margin of error is possible in the assignment of 2 theta angles and d-spacings, the preferred method of comparing X-ray powder diffraction patterns in order to identify a particular crystalline form is to overlay the X-ray powder diffraction pattern of the unknown form over the X-ray powder diffraction pattern of a known form. For all analytical data discussed in this application, it should be kept in mind that specific values depend on many factors, e.g., specific instrument, sample preparation and individual operator.

All TGA curves obtained from the present invention were carried out in a TGAQ500 of TA instruments (Lukens Drive, Del., USA). The thermogram was recorded from 40 to 150° C. under the nitrogen gas purge at a flow of 40 mL/min for balance and 60 mL/min for sample at a heating rate of 5° C./min. Differential scanning calorimetric analysis was carried out on TAQ1000. The thermogram was recorded from 40 to 150° C. under the nitrogen flow of 50 mL/min at a heating rate of 5° C./min. Weigh about 3-4 mg sample into aluminum pan and the sample was distributed uniformly as a thin layer. Polarizing light microscopy (hereinafter referred to as PLM) images were captured on Nikon Eclipse, 80/polarizing light microscope with a magnification of 50× to find particle shape.

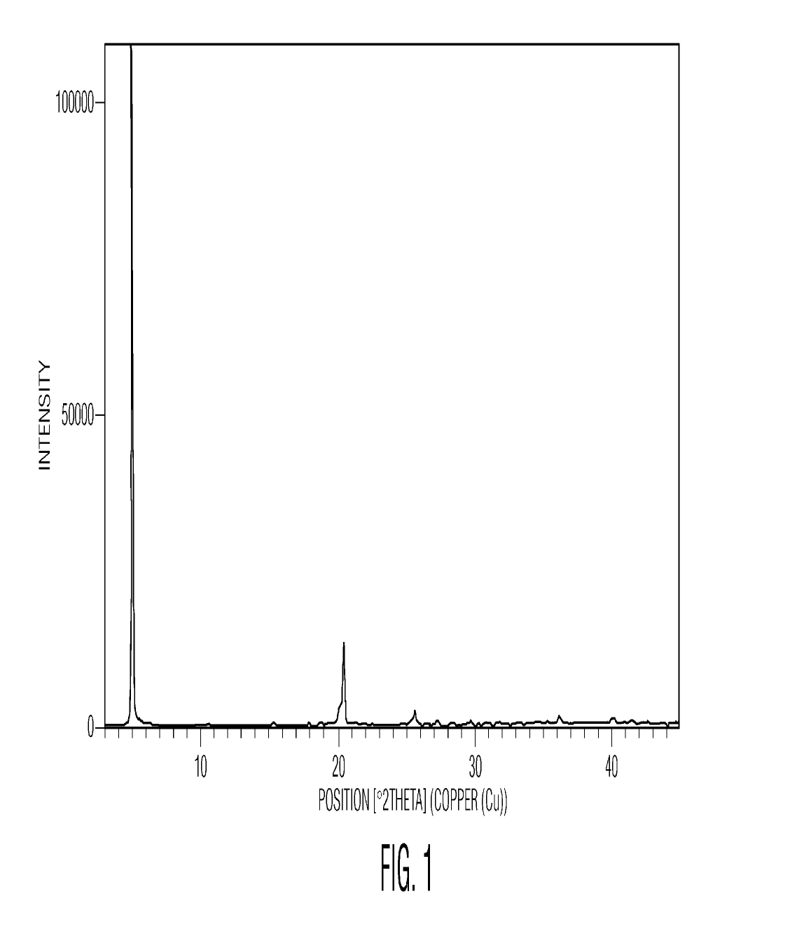

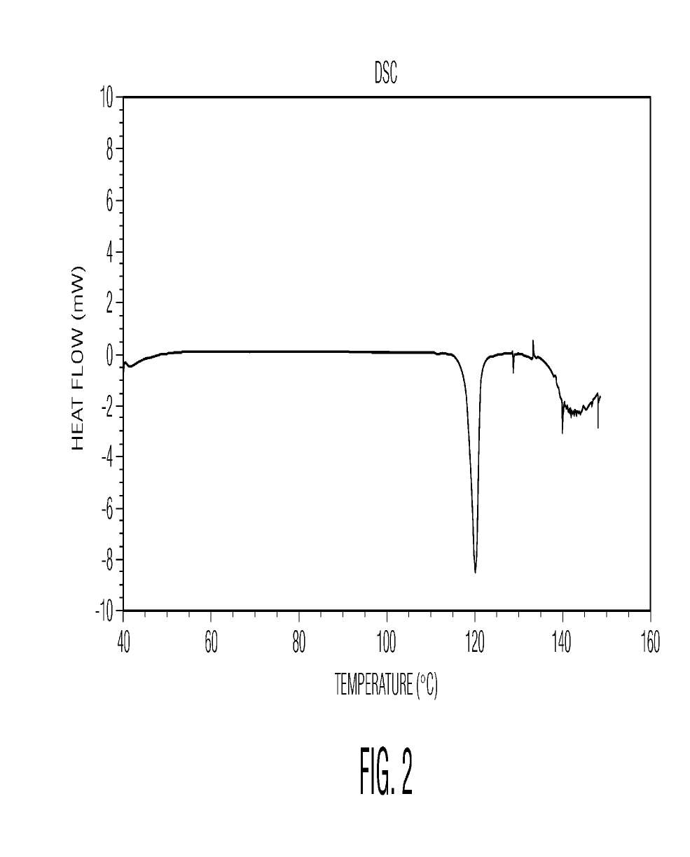

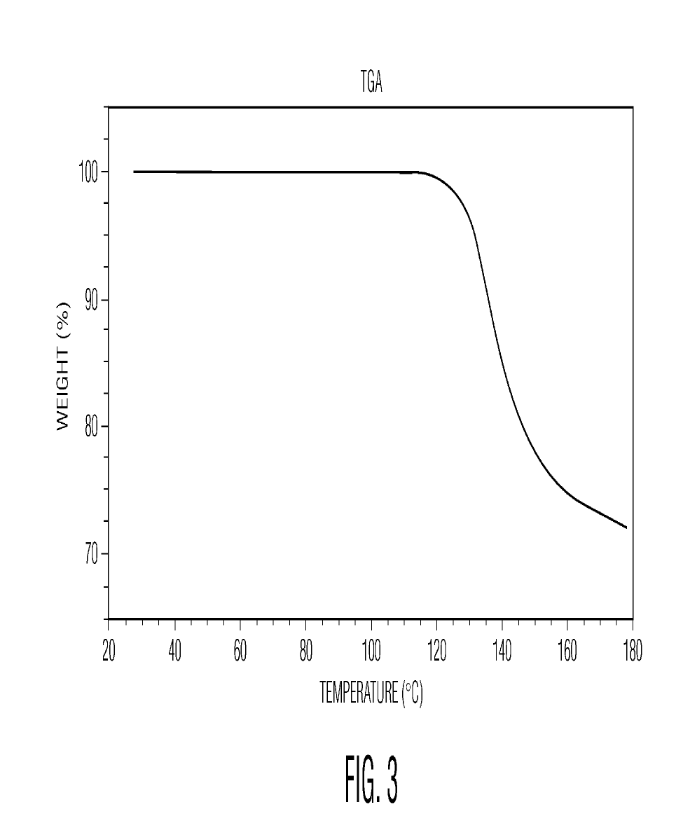

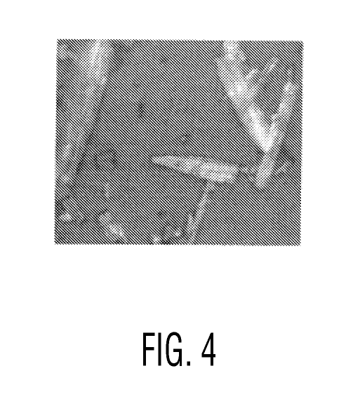

In one embodiment, the Capecitabine obtained from above processes, or otherwise, has an XRPD pattern substantially in accordance with FIG. 1. This form of Capecitabine is characterized by its DSC thermogram, which is shown in FIG. 2, having endothermic peaks at about 119.8° C. This Capecitabine has a characteristic thermo gravimetric (TGA) curve corresponding shows apparently no loss in the weight up to 100° C., as shown in FIG. 3. This indicates that the Capecitabine obtained from the present invention is anhydrous. Capecitabine is still further characterized by its PLM, which shows long needle morphology and depicted in FIG. 4. Capecitabine produced by this process (before Micronization) has shown a mean particle size of D₉₀ less than about 100 microns, D₅₀ less than about 50 microns, and D₁₀ less than about 10 microns.

In one embodiment, there is provided a method of producing solid particles of reduced median particle size or particle diameter, which comprises milling the solid in micronizer to obtain fine particles. Micronizer was set with required pressure at source for feeding as 2-5 Kgs/cm² and set the feeding pressure as 3-4 Kgs/cm². Milling or micronization can be performed prior to drying, or after the completion of drying of the product. Under the predefined conditions, the milling operation reduces the size of particles (diameter) to the desired level and increases surface area of particles. The mechanism of the same involves collision of particles with each other at high velocities at constant rates with predefined set conditions of milling. Milling is done suitably using jet milling equipment like an air jet mill, or using other conventional milling equipments.

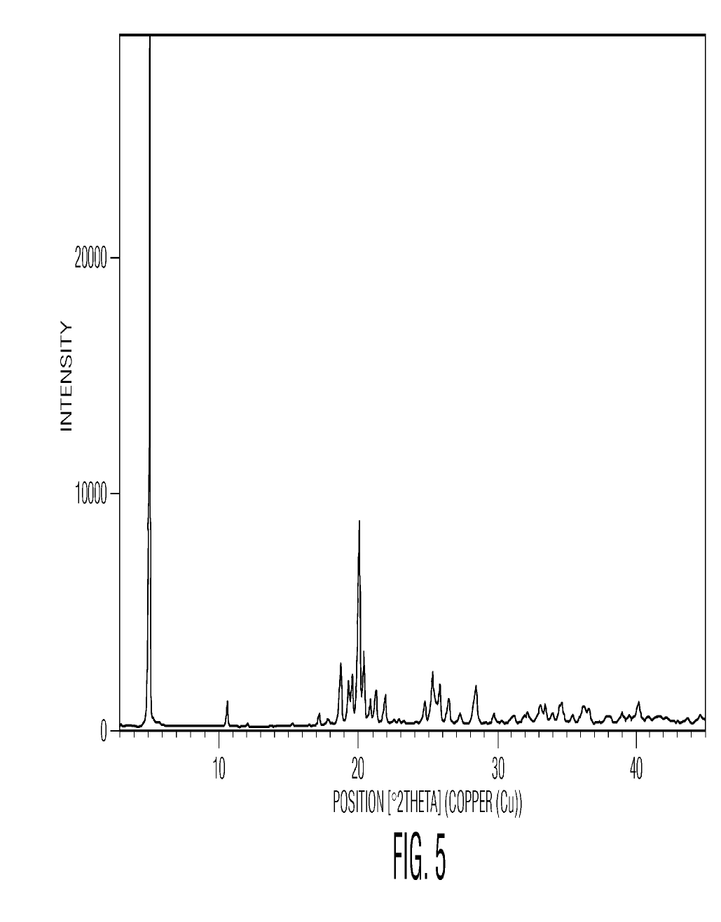

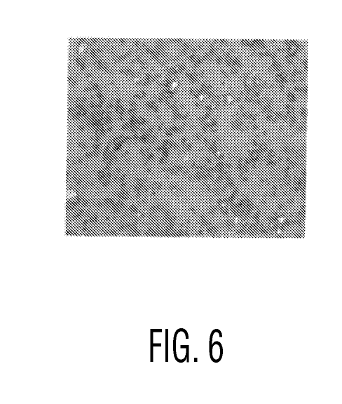

The Capecitabine obtained after Micronization having XRPD pattern substantially in accordance with FIG. 5. Capecitabine is still further characterized by its PLM, which shows smaller particles morphology and depicted in FIG. 6.

The final residual solvent level is preferably about 1 wt % or less, more preferably about 0.1 wt % or less.

In another embodiment Capecitabine obtained by the process of present invention, after milling, has a mean particle size D₉₀ of less than about 25 microns and/or D₅₀ of less than about 15 microns and/or D₁₀ of less than about 10 microns. A D₉₀, of less than about 25 microns, a D₅₀ of less than about 15 microns and a D₁₀ of less than about 10 microns as a particle size distribution has shown desirable dissolution profile in the preparation of pharmaceutical composition. In other embodiments, the mean particle size of the micronized compound of Formula I has D₉₀ of less than about 100 microns, a D₅₀ of less than about 50 microns and a D₁₀ of less than about 25 microns. In still another embodiment, the micronized compound of Formula I has a mean particle size of D₉₀ of less than about 10 microns, and/or a D₅₀ of less than about 10 microns and a D₁₀ of less than about 5 microns (falls through a 0.5 micron screen) are contemplated.

The D₁₀, D₅₀ and D₉₀ values are useful ways for indicating a particle size distribution. D₉₀ refers to the value for the particle size for which at least 90 volume percent of the particles have a size smaller than the value. Likewise D₅₀ and D₁₀ refer to the values for the particle size for which 50 volume percent, and 10 volume percent, of the particles have a size smaller than the value. Methods for determining D₁₀, D₅₀ and D₉₀ include laser diffraction, such as using laser light scattering equipment from Malvern Instruments Ltd. of Malvern, Worcestershire, United Kingdom. There is no specific lower limit for any of the D values.

In another embodiment, there is provided a pharmaceutical composition comprising Capecitabine produced by the processes of the present invention with at least one pharmaceutically acceptable excipient.

The pharmaceutical composition can be formulated as a liquid composition for oral administration including for example solutions, suspensions, syrups, elixirs and emulsions, containing inert diluents solvents or vehicles such as water, sorbitol, glycerine, propylene glycol or liquid paraffin, may be used.

Compositions for parenteral administration can be suspensions, emulsions or aqueous or non-aqueous, sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Solid oral dosage forms such as filled hard gelatin capsules, compressed tablets, gel caps where the capecitabine is suspended, dissolved, dispersed or emulsified in a vehicle surrounded by a soft capsule material are also contemplated. In these “solid” oral dosage forms, the capecitabine can be mixed with pharmaceutically acceptable excipients and/or solvent vehicles as described above.

Pharmaceutically acceptable excipients that are of use in the present invention include but are not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinyl pyrrolidone, hydroxypropyl cellulose, hydroxypropyl methyl cellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, complex forming agents such as various grades of cyclodextrins, resins; release rate controlling agents such as hydroxypropyl cellulose, hydroxymethyl cellulose, hydroxypropyl methyl cellulose, ethyl cellulose, methyl cellulose, various grades of methyl methacrylates, waxes and the like. Other pharmaceutically acceptable excipients that are of use include but not limited to film formers, plasticizers, colorants, flavoring agents, sweeteners, viscosity enhancers, preservatives, antioxidants and the like.

The dose used will depend upon a number of factors including, without limitation, the age of the patient, their health, the type of cancer, its extent and/or its location, the size of the patient and/or their surface area, and the sound discretion of the medical professional. However, daily doses of 1,000 mg/m²/day, 1,500 mg/m²/day, 1,750 mg/m²/day, 1,875 mg/m²/day, 2,000 mg/m²/day, 2,500 mg/m²/day, 3,000 mg/m²/day, 4,000 mg/m²/day, and 5,000 mg/m²/day are contemplated. These are given in one or more daily doses, usually two doses divided by 12 hours. Dosage forms may contain 100, 150, 200, 250, 500, 1000, 2000 mg per dosage form of capecitabine.

Certain specific aspects and embodiments of the invention will be explained in more detail with reference to the following examples, which are provided by way of illustration only and should not be construed as limiting the scope of the invention in any manner.

### EXAMPLES

### Example 1

### PREPARATION OF 2,3-O-ISOPROPYLIDENE-5-DEOXY-D-RIBOSE (FORMULA IV)

5-deoxy-D-ribose of Formula V (15 g), N,N-dimethylformamide (DMF; 60 ml), p-toluene sulfonic acid (385 mg) and 2,2-dimethoxy propane (30 ml) were charged into a clean and dry 4 neck round bottom flask. The resultant reaction mixture was stirred at 25-30° C. for 14 hours. Thin layer chromatography (“TLC”) was used to determine consumption of D-ribose. After completion of the reaction, the reaction mixture was distilled completely at 40° C. under a reduced pressure of about 600 mm Hg. Demineralized water (25 ml) was charged to the concentrated reaction mixture and stirred at 25-30° C. for 10 minutes. The pH of the reaction mixture was adjusted to about 6.8 using 5 ml of 20% sodium carbonate solution and then 100 ml of ethyl acetate was charged to the reaction mixture. The reaction mixture was stirred for 15 minutes and the organic and aqueous layers were separated. The aqueous layer was extracted with 20 ml of ethyl acetate. Both the organic layers were combined and the total organic layer was washed with 25 ml of water. Organic and aqueous layers were separated and the organic layer was dried over anhydrous sodium sulphate. The obtained organic layer was concentrated at 40° C. under vacuum to dryness, affording 12.8 g of the title compound.

H′NMR: δ values: 1.33 (d, 3H), 1.45 (s, 6H), 4.2 (q, 1H), 4.5 (d, 1H), 4.6 (d, 1H), 5.3 (s, 1H)

MASS: m/z 192.4 (m⁺H)+NH₃

### Example 2

### PREPARATION OF 2,3-O-ISOPROPYLIDENE-3-O-ACETYL-5-DEOXY-D-RIBOSE (FORMULA III)

2,3-O-isopropylidene-5-deoxy-D-ribose of Formula IV (12.8 g) and pyridine (51.2 ml) were charged into a clean and dry 4 neck round bottom flask followed by cooling to 0-5° C. Acetic anhydride (10.24 ml) was added over about 40 minutes at 0-5° C. and then the reaction mixture was heated to 42° C. The resultant reaction mixture was stirred at 42° C. for 1.5 hours. TLC was used to determine the conversion of 2,3-O-isopropylidene-5-deoxy-D-ribose. After completion of the reaction, the reaction mixture was cooled to 25-30° C. and the pH was adjusted to 6.5 using 7% aqueous sodium bicarbonate solution (300 ml). The reaction mixture was extracted with dichloromethane (2×150 ml) followed by separation of organic and aqueous layers. The organic layer was washed with 10% aqueous hydrochloric acid solution (2×175 ml) followed by washing with saturated sodium chloride solution (150 ml). Organic and aqueous layers were separated and the organic layer was washed with water (2×175 ml) followed by separation of organic and aqueous layers. The organic layer was dried over anhydrous sodium sulfate. Finally, the organic layer was distilled completely at 40° C. under a vacuum of about 600 mm Hg. The obtained concentrated reaction residue was stripped with n-heptane (2×50 ml) to afford 11.4 g of the title compound.

H′NMR: δ values: 1.33 (d, 3H), 1.45 (s, 6H), 2.05 (s, 3H), 4.4 (q, 1H), 4.5 (d, 1H), 4.7 (d, 1H), 6.2 (s, 1H)

MASS: m/z 234.0 (m⁺H)+NH₃

### Example 3

### PREPARATION OF N-[(PENTYLOXY)CARBONYL]-5-FLUOROCYTOSINE (FORMULA IIIA)

5-fluorocytidine (10 g) was charged into a clean and dry 4 neck round bottom flask followed by charging of pyridine (60 ml). n-pentyl chloroformate (5.9 ml) was added to the reaction mixture at 25 to 30° C. over 10 minutes. After completion of the addition, the reaction solution was heated to 100-110° C. followed by stirring for 2 hours. Conversion of 5-fluorocytidine was monitored by TLC. After completion of the reaction, the reaction solution was allowed to reach a temperature of 25-30° C. The reaction solution was filtered and the filtrate was charged into a flask containing water (200 ml) followed by stirring for 20 minutes. The suspension obtained was filtered and the solid was washed with isopropyl alcohol (50 ml). The solid obtained was subjected to suction at about 600 mm Hg to afford 10.5 g of the title compound.

### Example 4

### PREPARATION OF 5′-DEOXY-2′,3′-O-ISOPROPYLIDENE-N-[(PENTYLOXY)CARBONYL]-5-FLUOROCYTIDINE (FORMULA II)

N-[(pentyloxy)carbonyl]-5-fluorocytosine of Formula IIIA (2.65 g) was charged into a flask followed by charging of hexamethyldisilazane (HMDS; 15 ml) and trimethylsilylchloride (TMS-Cl; 0.06 ml). The reaction mixture was heated to 80° C. and stirred for 2 hours. The resultant reaction solution was cooled to 50° C. and then the reaction mixture was striped twice with toluene (25 ml), and then cooled to 25-30° C. to afford silylated N-[(pentyloxy)carbonyl]-5-fluorocytosine of Formula IIIB.

Dichloromethane (30 ml) and 2,3-O-isopropylidene-3-O-acetyl-5-deoxy-D-ribose of Formula III (2.5 g) were charged to the above obtained silylated N-[(pentyloxy)carbonyl]-5-fluorocytosine compound of Formula IIIB. The reaction mixture was stirred at 25-35° C. for 10 minutes and then cooled to 0-5° C. Stannic chloride (1.6 ml) was added over 10 minutes to the above reaction mixture at 0-5° C. and stirred for 2 hours. The temperature of the reaction mixture was raised to 25-30° C. and stirred for 1 hour. Conversion was monitored using TLC. After completion of the reaction, the reaction was decomposed by the charging of sodium bicarbonate (5 g) and stirred at 25-30° C. for 1 hour. The reaction suspension was filtered and then the obtained filtrate was separated into two layers. The aqueous layer was extracted with dichloromethane (2×50 ml) followed by separation of organic and aqueous layers. Both the organic layers were combined and the total organic layer was washed with 5% aqueous hydrochloric acid solution (100 ml). Organic and aqueous layers were separated and the organic layer was washed with 10% aqueous hydrochloric acid (100 ml). Organic and aqueous layers were separated and the organic layer was washed with water (2×100 ml) and then the organic layer was distilled completely at 40° C. The residue obtained was purified by column chromatography using 30% of ethyl acetate in petroleum ether as the eluent to afford 1.23 g of the title compound.

MASS: m/z 399.9[m⁺+H]

NMR: δ values: 0.88 (t, 3H), 1.29-1.6 (m, 6H), 1.33 (d, 3H), 1.45 (s, 6H), 1.6 (t, 2H), 4.09 (q, 1H), 4.55 (d, 1H), 5.0 (d, 1H), 5.7 (s, 1H), 8.2 (d, 1H), 10.6 (s, NH).

### Example 5

### PREPARATION OF CAPECITABINE (FORMULA I)

5′-deoxy-2′,3′-O-isopropylidene-N-[(pentyloxy)carbonyl]-5-fluorocytidine of Formula II (460 mg), obtained in Example 4, absolute ethanol (22 ml), Amberlyst™ 15 catalyst (3.5 g) and demineralized water (0.6 ml) were charged into a clean and dry 4 neck round bottom flask followed by stirring for 9 hours at 25-30° C. Conversion of 5′-deoxy-2′,3′-O-isopropylidene-N-[(pentyloxy)carbonyl]-5-fluorocytidine was monitored by TLC. After completion of the reaction, the reaction mixture was filtered through a celite bed and the celite was washed with ethanol (5 ml). The filtrate obtained was distilled completely at 40° C. Diisopropyl ether (10 ml) was added to the residue and stirred at 25-30° C. for 15 minutes, and distilled completely at 40° C. under a vacuum of 600 mm Hg. Ethyl acetate (1.5 ml) was added to the residue and cooled to 0-5° C. The solution was stirred for 30 minutes. n-hexane (2 ml) was charged, solid was precipitate and stirred at 25-30° C. for 30 minutes. The solid that formed was filtered and the solid was washed with precooled ethyl acetate (0.5 ml) to afford 0.12 g of the title compound.

NMR: 0.88 (t, 3H), 1.291.6 (m, 6H), 1.33 (d, 3H), 3.67 (q, 1H), 3.86 (t, 1H), 3.86 (s, 1H), 4.09 (t, 2H), 5.07 (d, 1H), 5.43 (d, 1H), 8.06 (s, OH), 10.56 (t, OH), 11.65 (s, NH).

Mass: m/z 359.9[m⁺+H]

### Example 6

### PREPARATION OF 5′-DEOXY-2′,3′-O-ISOPROPYLIDENE-5-FLUOROCYTIDINE (FORMULA VI)

5-fluoro cytosine (0.58 g), hexamethyldisilazane (HMDS; 0.95 ml), trimethylsilylchloride (TMS-Cl; 0.1 ml), and toluene (6 ml) were charged into a clean and dry 4 neck round bottom flask under a nitrogen atmosphere. The reaction mixture was heated to 110-120° C. under a nitrogen atmosphere followed by stirring for 30 minutes. The reaction solution was cooled to 55-65° C. and the solvent toluene was distilled completely under a vacuum of 600 mm Hg. The residue was cooled to 25-30° C. under a nitrogen atmosphere and dichloromethane (10 ml) was charged to the residue. The obtained reaction residue (silylated compound) was cooled to 0-5° C. Dissolved 1 g of 2,3-O-isopropylidene-3-O-acetyl-5-deoxy-D-ribose of Formula III in dichloromethane (2 ml) and then added dropwise to the above silylated residue at 0-5° C. over 10 minutes. Stannic chloride (0.6 ml) was charged to the above reaction suspension at 0-5° C. The reaction solution obtained was allowed to reach a temperature of 25-30° C. followed by stirring for 2 hours. Conversion of the reactants to product was monitored by TLC. After completion of the reaction, sodium bicarbonate (1.6 g) was charged to above reaction solution and then demineralized water (0.6 ml) was added. The reaction suspension was stirred at 25-30° C. for 2 hours and the suspension was filtered through a celite bed and the filtrate was washed with 5% aqueous sodium bicarbonate solution (10 ml). The organic solution was dried over anhydrous sodium sulfate and then distilled completely at 45° C. under a vacuum of 600 mm Hg. The residue obtained was purified by column chromatography using 10% methanol in dichloromethane as eluent to afford 0.4 g of the title compound.

Mass: m/z 286.2[m⁺+H]

H′NMR: δ 1.33 (d, 3H), 1.45 (s, 6H), 4.01 (q, 1H), 4.7 (d, 1H), 4.9 (d, 1H), 5.7 (s, 1H), 7.5-7.8 (2H), 7.9 (d, 1H)

### Example 7

### PREPARATION OF 5′-DEOXY-2′,3′-O-ISOPROPYLIDENE-N-[(PENTYLOXY)CARBONYL]-5-FLUOROCYTIDINE (FORMULA II)

5′-deoxy-2′,3′-O-isopropylidene-5-fluorocytidine of Formula VI (5.5 g) and dichloromethane (19.25 ml) were charged into a clean and dry 4 neck round bottom flask followed by stirring for 5 minutes. Pyridine (3.14 ml) was charged to the above reaction mixture followed by cooling to −10 to −15° C. n-pentyl chloroformate (5.9 ml) was added to the reaction solution over 2 hours. The resultant reaction solution was allowed to reach the temperature to 25-30° C. and was stirred for 30 minutes. After completion of the reaction, methanol (0.35 ml), dichloromethane (22 ml) and water (11 ml) were charged to the reaction mixture. The reaction suspension was stirred for 15 minutes followed by separation of organic and aqueous layers. The organic layer was washed with water (1 ml) followed by drying the organic layer over anhydrous sodium sulfate. The organic layer was distilled completely at 40° C. under a vacuum of 600 mm Hg to afford 7 g of the title compound.

MASS: m/z 399.9[m⁺+H]

NMR: δ values: 0.88 (t, 3H), 1.29-1.6 (m, 6H), 1.33 (d, 3H), 1.45 (s, 6H), 1.6 (t, 2H), 4.09 (q, 1H), 4.55 (d, 1H), 5.0 (d, 1H), 5.7 (s, 1H), 8.2 (s, 1H), 10.6 (s, NH).

### Example 8

### PREPARATION OF CAPECITABINE (FORMULA I)

5′-deoxy-2′,3′-O-isopropylidene-n-[(pentyloxy)carbonyl]-5-fluorocytidine of Formula II (5.5 g), obtained in Example 7, was charged into a clean and dry 4 neck round bottom flask. Ethanol (110 ml) and water (5 ml) were charged followed by stirring for about 10 minutes. Amberlyst™ 15 catalyst (5.5 g) was charged to the reaction solution and stirred for 8 hours. After completion of the reaction, the reaction mixture was filtered and then the obtained filtrate was distilled completely at 45° C. under a vacuum of 600 mm Hg. Ethyl acetate (9.3 ml) was charged to the residue and heated to 45-50° C. for 15 minutes. The solution was cooled to 25-30° C. and stirred for 30 minutes. The solution was further cooled to 0° C. and the suspension was stirred for 1 hour. The solid that formed was filtered and the solid was washed with precooled ethyl acetate (3 ml). The solid obtained was dried at 35° C. under a vacuum of 600 mm Hg for 4 hours to afford 1 g of the title compound.

### Example 9

### PROCESS FOR PREPARING CAPECITABINE OF FORMULA I

5′-deoxy-2′,3′-O-acetyl-5-fluorocytidine (33 kg) and dichloromethane (115.5 lit) were charged into the reactor at room temperature and stirred for 10 minutes. Pyridine (16.5 lit) was charged to the obtained reaction mass and applied nitrogen gas to the reactor followed by cooling to −10 to −15° C. by using methylene glycol solution into the jacket of the reactor. n-pentyl chloroformate (33 lit) was added slowly to the reaction mixture for a period of 3 to 5 hours at temperature less than 5° C. and stirred for 1 hour 30 minutes. After completion of the reaction, methanol (2 lit) was charged to the reaction mixture at temperature between 0 to −5° C. and stirred the reaction mixture for 15 minutes. Dichloromethane (132 lit) and demineralized water (66 lit) were charged to the reaction mass and stirred for 5 minutes. Two layers were separated from the obtained solution. The obtained organic layer was dried with sodium sulphate (14.5 kg) and then concentrated at temperature less than 40° C. under vacuum not less than 650 mmHg till no more solvent distill off followed by applying nitrogen gas to remove the pyridine traces. The reaction crude was cooled to the temperature 30 to 35° C. and released the vacuum with nitrogen.

Purity: 96.4% by HPLC.

Methanol (99 lit) was charged to the resultant reaction crude obtained from above process at 25-30° C. and stirred for 30-45 minutes. The reaction solution was cooled to −5 to −15° C. Sodium hydroxide solution (obtained by dissolution of 6.3 kg of sodium hydroxide in 158 lit of demineralized water) was added slowly to the reaction mixture at temperature −5 to −15° C. for a period of 1½ to 2 hours under nitrogen atmosphere followed by stirring for 15 minutes. Hydrochloric acid (17 lit) was added drop wise to the reaction mass to adjust the pH between 4 and 5 at temperature −5 to −15° C. The reaction mass was allowed to raise the temperature to 25-30° C. followed by addition of dichloromethane (297 lit) to the reaction mass and stirred the whole reaction mixture for 15 minutes. Two layers were separated and the obtained organic layer was washed with demineralized water (99 lit). Sodium sulphate (19 kg) was charged to the organic layer and stirred for 5 minutes and then allowed to settle for 15 minutes. The reaction solution was filtered and washed the cake with dichloromethane (30 lit). To the filtrate, activated carbon (5 kg) was added and stirred the whole solution for 15 minutes. The resultant solution was filtered on hyflow super cell and washed the hyflow super cell bed with dichloromethane (30 lit). The total filtrate was concentrated at a temperature below 45° C. under vacuum between not less than 600 mmHg till no more solvent distills off. The reaction crude was cooled to the temperature 30 to 35° C. and dissolved in ethyl acetate (74 lit). The reaction solution was allowed to raise the temperature to 30-35° C. and stirred the reaction mixture for 15 minutes. n-hexane (111.5 lit) was charged to the reaction mixture and cooled to 15-20° C. followed by stirring for 1 hour. The reaction mixture was subjected to centrifuge and then washed the wet cake with mixture of ethyl acetate and n-hexane (14.6 lit+22.5 lit) followed by washing with n-hexane (25 lit). The obtained solid was dried at temperature 35 to 40° C. under vacuum not less than 650 mmHg for 12 hours to obtain 22.6 kg of title compound.

### Seiving:

Capecitabine (25 kg) was charged into container, which was arranged with shifter. The material was sieved through shifter and then weighed to obtain 22.5 kg.

XRPD pattern—As shown in FIG. 1

DSC: 119.84° C.

TGA: no weight loss up to 100° C.

Particle size distribution:
D₁₀: 1.82 μm
D₅₀: 5.53 μm
D₉₀: 40.51 μm

Water content: 0.06% by Karl Fisher method (KF method)

Purity: 99.6% w/w assay by HPLC. (Single impurity: 0.005%; Total impurities: 0.15%)

Micronization:

Capecitabine (3.9 kg), obtained according to above process, was charged into micronizer. Micronization was started slowly through the product feed funnel for micronizer through the hopper at the feed rate of 2 to 3 kgs/hour and the material was collected into the collector at feed pressure 3-4 kgs/cm2. Finally the collector was removed from the micronizer and the material was unloaded to obtain 3.83 kgs.

XRPD pattern has shown in FIG. 5

DSC: 120.07° C.

TGA: no weight loss up to 100° C.

Particle size distribution:
10% less than 1.08 μm
50% less than 2.46 μm
90% less than 5.04 μm

Water content: 0.05% w/w

Purity: 99.8% assay by HPLC. (Single maximum impurity: 0.05%; Total impurities: 0.13%)

### Example 10

### PROCESS FOR PREPARING CAPECITABINE OF FORMULA I

5′-deoxy-2′,3′-O-acetyl-N-[(pentyloxy)carbonyl]-5-fluorocytidine of Formula C (20 g) was dissolved in methanol (40 ml) and cooled the whole solution to −10 to −15° C. 1 N sodium hydroxide was added to the obtained reaction solution over a period of 30 minutes at a temperature of −5 to −10° C. After completion of the reaction, the reaction mixture was adjusted pH to 4.24 by using conc.hydroxhloric acid (6.1 ml). Dichloromethane (200 ml) was charged to the reaction mixture followed by two layers were separated. The resultant organic layer was washed with demineralized water (100 ml) and then concentrated the organic layer up to reach 2 volumes (32 ml) of the solvent in the reaction solution. Then the reaction solution was cooled to room temperature. Toluene (160 ml) was charged to the reaction solution and stirred for 2 to 3 hours and then the suspension was filtered. The solid was washed with toluene (16 ml) and then dried for 4 to 5 hours at 40° C. to afford 11.4 g of title compound.

Purity: 99.74% by HPLC.

1. A process for the preparation of the compound of Formula I 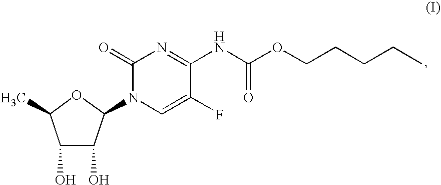
comprising deprotecting a compound of Formula II, 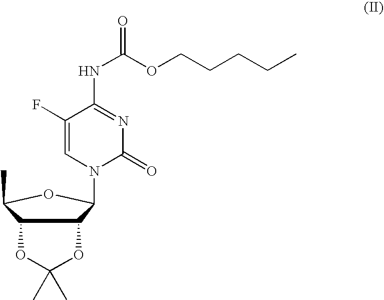
by treating with Amberlyst™ 15 resin.

2-3. (canceled)

4. The process according to claim 1, wherein the amount of Amberlyst™ 15 resin in the conversion of Formula II to Formula I ranges from about 0.5 to about 2 times the weight of the compound of Formula II.

5. The process according to claim 1, wherein the conversion of Formula II to Formula I is conducted in an aqueous alcohol solvent.

6-17. (canceled)

18. A process for preparing the compound of Formula II, 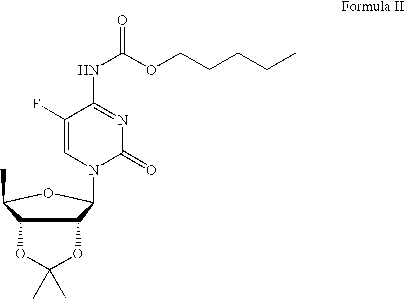
comprising:
a) reacting 5-deoxy-D-ribose of Formula V: 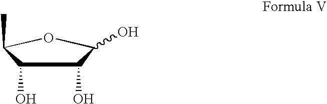
with 2,2-dimethoxypropane in an organic solvent to afford 2,3-O-isopropylidene-5-deoxy-D-ribose of Formula IV; 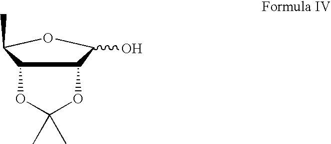
b) reacting 2,3-O-isopropylidene-5-deoxy-D-ribose of Formula IV with acetic anhydride in the presence of an organic solvent to afford 2,3-O-isopropylidene-1-O-acetyl-5-deoxy-D-ribose of Formula III 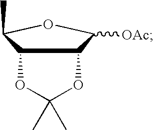
and
c) reacting 2,3-O-isopropylidene-1-O-acetyl-5-deoxy-D-ribose of Formula III with 2-O-trimethyl silyl, N-[(pentyloxy)carbonyl]-5-fluorocytosine of Formula IIIB: 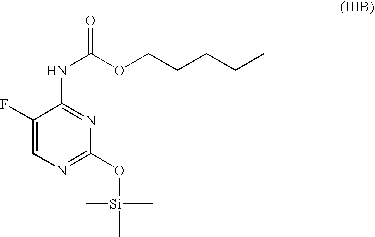
in the presence of an organic solvent to afford 5′-deoxy-2′,3′-O-isopropylidene-N-[(pentyloxy)carbonyl]-5-fluorocytidine of Formula II.

19. The process according to claim 18, wherein step a) is conducted in the presence of an acid selected from para-toluene sulfonic acid, oxalic acid, tartaric acid, formic acid, acetic acid, hydrochloric acid, and sulphuric acid.

20. The process according to claim 18, wherein step b) is conducted in presence of a base selected from the group consisting of pyridine, triethylamine, methylamine, sodium hydroxide, potassium hydroxide, and lithium hydroxide.

21. A process for preparing 5′-deoxy-2′,3′-O-isopropylidene-N-[(pentyloxy)carbonyl]-5-fluorocytidine of Formula II, comprising:
i) reacting 2,3-O-isopropylidene-3-O-acetyl-5-deoxy-D-ribose of Formula III: 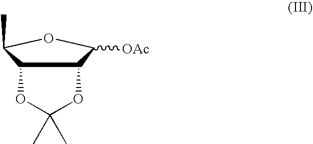
with 2-O-trimethyl silyl, N-(trimethyl silyl)-5-fluorocytosine of Formula IIIC: 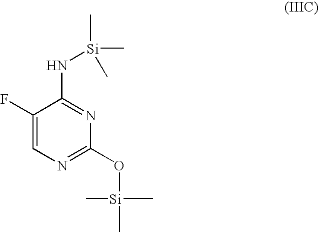
in the presence of stannic chloride and an organic solvent to afford 5′-deoxy-2′,3′-O-isopropylidene-5-fluorocytidine of Formula VI 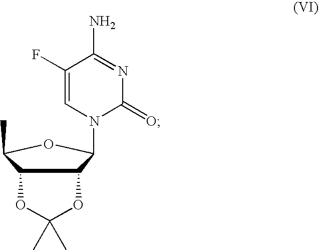
and
ii) reacting 5′-deoxy-2′,3′-O-isopropylidene-5-fluorocytidine of Formula VI with n-pentyl chloroformate in the presence of an organic solvent to afford the compound of Formula II.

22. The process according to claim 21, wherein the organic solvent of step i) is selected from a chlorinated hydrocarbon or a hydrocarbon or any mixtures thereof.

23. The process according to claim 21, wherein the amount of stannic chloride used in step i) ranges from about 0.5 to about 2 molar equivalents per molar equivalent of the compound of Formula III.

24. The process according to claim 21, wherein said organic solvent for step ii) is selected from a halogenated hydrocarbon, a hydrocarbon, an ether, and any mixture thereof.

25-27. (canceled)
